(19) United States
(12) Patent Application Publication
(10) Pub. No.: US 2004/0170689 A1
(43) Pub. Date: Sep. 2, 2004

(54) Stabilized formulations comprising hydrolytically unstable compositions

(76) Inventors: Debra Alida Odink, Mountain View, CA (US); I-Lan Sue, San Jose, CA (US); Gary Conard Visor, Belmont, CA (US)

Correspondence Address: ROCHE PALO ALTO LLC, PATENT LAW DEPT. M/S A2-250, 3431 HILLVIEW AVENUE, PALO ALTO, CA, 94304, US

(21) Appl. No.: 10/798,856

(22) Filed: Mar. 11, 2004

Related U.S. Application Data
(62) Division of application No. 10/037,875, filed on Nov. 9, 2001.

(51) Int. Cl. A61K 9/46; A61K 9/24

(52) U.S. Cl. 424/471; 514/401

(57) ABSTRACT

This invention relates to new stable oral pharmaceutical formulations prepared by covering an nucleus formed by a core with a first hydrophobic polymer layer, a second layer coating the first layer, wherein said second layer comprises one or more labile pharmaceutical compounds in one or more acceptable hydrophobic excipients, and an optional third enteric polymer layer.

### CROSS-REFERENCE TO RELATED APPLICATIONS

This application claims benefit under Title 35 U.S.C. 119(e) of U.S. Provisional Application Nos. 60/247,257 filed Nov. 10, 2000 and 60/326,274 filed Oct. 1, 2001.

### FIELD OF THE INVENTION

This invention relates to stable formulations comprising hydrolytically unstable pharmaceutically active compounds, in particular certain formulations comprising hydrolytically unstable compounds with an imidazoline moiety.

The invention also relates to a process for the production thereof and a method for the treatment of urinary incontinence.

### BACKGROUND OF THE INVENTION

The pharmaceutical industry employs a variety of dosage formulations for orally administering medicinal agents to patients. An important aspect of the manufacture, regulatory review, and approval of all dosage forms concerns their stability over extended periods of time. It is well recognized that the humidity content of the product can influence its stability. Therefore precautions must be taken not to alter the product in the effort to obtain stabilized formulations, by insuring that processing does not change the product with the introduction of humidity.

The use of a barrier layer to protect the pharmaceutically active compound from degradation caused by the enteric coating or by the environment is well known in the art (as described, for example, in U.S. Pat. No. 5,626,875). It is also well known to use a core which is coated with a pharmaceutical compound in conjunction with a binder agent (as described, for example, in EP 519,144). Other references also deal with stability problems by incorporating stabilizing excipients to the formulation (as described, for example, in WO 94/407493 or in U.S.Pat. No. 4,743,450). To date, stability problems caused by direct contact or interaction of labile therapeutically active drugs with ingredients of the core, resulting in degradation of the drug, have not yet been addressed. Particularly stability problems of imidazoline drugs may arise when the compound comes in contact with humidity in the presence of the core. Stability problems in this context have not been addressed.

In the case of certain formulations comprising an active compound at very low dosages (e.g. an imidazoline moiety) and conventional excipients, degradation of the active compound was observed. It was found that, although not hygroscopic, the compound was unstable and underwent hydrolysis in the conventional environments of solid formulations involving solid cores, e.g., beads. The present invention has the advantage of isolating the core from the active pharmaceutical compound with an enteric polymer layer providing an acidic micro environment, which may result in greater stability of the labile pharmaceutical composition.

The object of the present invention is therefore directed to a pharmaceutical formulation which reduces the degradation of the pharmaceutically active compound.

The object of the present invention is also directed to a low dose mixture which comprises a uniform particulate consistency throughout the formulation.

### DESCRIPTION OF THE RELATED ART

U.S. Pat. No. 5,626,875 assigned to Esteve Quimica refers to certain stabilized galenic formulations comprising an acid labile benzimidazole compound.

WO 94/07493 assigned to Warner-Lambert Co. refers to certain stabilized formulations containing the cognition activator CI-979 HCI comprising adipic acid as an excipient.

U.S. Pat. No. 5,362,860 assigned to Warner-Lambert Co. refers to a certain neutral stabilization complex for CI-979 HCI by formation of a complex with cyclic polydextrose.

U.S. Pat. No. 4,743,450 assigned to Warner-Lambert Co. refers to a certain stabilized formulation containing a metal-containing stabilizer and a saccharide.

U.S. Pat. No. 5,338,548 assigned to Parmetrix Co. refers to a certain method for increasing the storage stability of physostigmine by incorporating the free base into a polymer matrix.

U.S. Pat. No. 5,711,954 assigned to Schering-Plough HealthCare Products, Inc. refers to a certain stable powder formulation comprising an effective amount of an imidazole antifungal compound, and talc coated with a hydrophobic coating.

EP 519,144 assigned to Ilsan Ilac Ve Hammaddelelri Sanayi A.S. refers to a certain production method for enteric coated pellets containing Omeprazole which is coated on a core in the form of pH buffered dispersion phase.

All publications, patents, and patent applications cited herein, whether supra or infra, are each hereby incorporated by reference in its entirety.

### SUMMARY OF THE INVENTION

In one aspect, this invention relates to a stabilized oral pharmaceutical formulation comprising a nucleus formed by a core, a first layer that comprises an enteric polymer sealing the core, a second layer coating the first layer that comprises one or more pharmaceutically active compounds in one or more acceptable hydrophobic excipients.

In another embodiment a third layer that comprises an enteric polymer may coat the second layer to further stabilize the formulation, to prevent degradation by gastric fluid and enzymes, or to provide delayed or sustained release medication.

In another embodiment the first polymer layer is a hydrophobic enteric polymer selected from the group comprising acrylic polymers, alkylcelluloses, and mixtures thereof, more preferably the pharmaceutical formulation comprises the first polymer layer comprising a hydrophobic polymer selected from the group comprising shellac or Eudragit™, preferably series L or S.

In a preferred embodiment, the invention relates to galenic formulations wherein the labile pharmaceutically active compound is susceptible to hydrolytic degradation, more preferably the labile pharmaceutically active compound comprises an imidazoline moiety, even more preferably the labile pharmaceutically active compound has a Formula Ar—A—B, wherein Ar is a substituted aryl group, A is—NH—, —CH₂—or —OCH₂—, and B is 2-imidazoline. In another preferred embodiment the labile pharmaceutically active compound is a compound of Formula I:

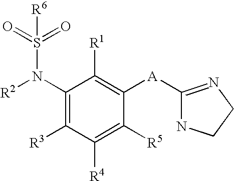

wherein:

A is —NH—, —CH₂—, or —OCH₂—;

R¹, R³, R⁴, and R⁵ are each independently in each occurrence hydrogen, (C₁-C₆) alkyl, or halogen;

R⁶ is (C₁-C₆) alkyl;

R² is hydrogen or (C₁-C₆) alkyl; or

R² and R³ taken together with the atoms to which they are attached may form a 5- or 6- membered ring;

in another preferred embodiment the labile pharmaceutically active compound is a compound of Formula I, wherein A is —OCH₂—, R¹ and R⁶ are methyl, R³ is chloro, and R², R⁴ and R⁵ are hydrogen, named N-[6-chloro-3-(4,5-dihydro-1H-imidazol-2-ylmethoxy) -2-methyl-phenyl]-methanesulfonamide; or pharmaceutically acceptable salts thereof.

Processes for the preparation of compounds of Formula I and of N-[6-chloro-3-( 4,5-dihydro-1H-imidazol-2-ylmethoxy)-2-methyl-phenyl]-methanesulfonamide are disclosed in U.S. Pat. No. 5,952,362.

Another aspect of this invention relates to a process for the manufacture of a formulation comprising one or more labile pharmaceutically active compounds, which comprises coating a core with a first layer sealing the core, wherein said first layer comprises an enteric polymer layer and optionally one or more hydrophobic excipients such as but not limited, to talc, in a non-aqueous solvent such as, but not limited to, dehydrated alcohol (200 proof); drying said first layer; coating said first layer with a second layer, wherein said second layer comprises one or more labile pharmaceutically active compounds suspended in one or more acceptable hydrophobic excipients in a non-aqueous solvent such as but not limited to, dehydrated alcohol (200 proof); drying the second layer; optionally coating the second layer with a third layer, wherein said third layer comprises an enteric polymer in a non-aqueous solvent such as but not limited to, dehydrated alcohol (200 proof), providing further stabilization, or allowing delayed or sustained release; and drying the third layer. In another preferred embodiment the pharmaceutically active compound is a compound of Formula I; and in another preferred embodiment the pharmaceutically active compound is a compound of Formula I, wherein A is —OCH₂—, R¹ and R⁶ are methyl, R³ is chloro, and R², R⁴ and R⁵ are hydrogen, named N-[6-chloro-3-(4,5-dihydro-1H-imidazol-2-ylmethoxy)-2-methyl-phenyl]-methanesulfonamide.

An additional aspect of the invention relates to a method of treatment of urinary incontinence comprising administering a stable oral pharmaceutical formulation comprising a nucleus formed by a core, a first layer, wherein said first layer comprises a hydrophobic enteric polymer layer sealing the core and optionally one or more excipients; a second layer coating the first layer, wherein said second layer comprises a pharmaceutically active compound of Formula I, wherein A is —OCH₂—, R¹ and R⁶ are methyl, R³ is chloro, and R², R⁴ and R₅ are hydrogen, named [6-chloro-3-(4,5-dihydro-1H-imidazol-2-ylmethoxy)-2-methyl-phenyl]-methanesulfonamide, in one or more acceptable hydrophobic excipients; in another preferred embodiment the invention relates to a method of treatment of urinary incontinence comprising administering a stable oral pharmaceutical formulation comprising a nucleus formed by a core, a first layer, wherein said first layer comprises a hydrophobic enteric polymer layer and optionally one or more hydrophobic excipients sealing the core, a second layer coating the first layer, wherein said second layer comprises a pharmaceutically active compound of Formula I, wherein A is —OCH₂—, R¹ and R⁶ are methyl, R³ is chloro, and R², R⁴ and R⁵ are hydrogen, named N-[6-chloro-3-(4,5-dihydro-1H-imidazol-2-ylmethoxy)-2-methyl -phenyl]-methanesulfonamide, in one or more acceptable hydrophobic excipients, and a third layer coating the second layer comprising an enteric polymer in a non-aqueous solvent providing further stabilization, or allowing delayed or sustained release.

In another embodiment, the method of treatment comprises administering the stable formulations in a capsule or pellet form.

### DETAILED DESCRIPTION OF THE INVENTION

Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon radical, having from one to six carbon atoms inclusive, unless otherwise indicated. Examples of lower alkyl radicals include, but are not limited to, methyl, ethyl, propyl, isopropyl, 1-ethylpropyl, sec-butyl, tert-butyl, n-butyl, n-pentyl, n-hexyl, and the like.

“Aryl” means the monovalent aromatic carbocyclic radical consisting of one individual ring, or one or more fused rings in which at least one ring is aromatic in nature, which can optionally be substituted with one or more, preferably one or two, substituents selected from hydroxy, cyano, lower alkyl, lower alkoxy, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, and arylcarbonylamino, unless otherwise indicated. Alternatively two adjacent atoms of the substituents taken together with the atoms to which they are attached may also form a 5- to 7- member ring. Examples of aryl radicals include, but are not limited to, phenyl, naphthyl, indanyl, 3-methanesulfonylamino-phenyl, 2-methyl-3-methanesulfoonylamino -4-chloro-phenyl; 2-methyl-3-methanesulfonylamino-4-bromo-phenyl, and the like.

“Halogen” means the radical fluoro, bromo, chloro, and/or iodo.

“Excipient” means any inert component admixed with or co-incorporated with the therapeutically active agent onto the surface of or into the substrate. Excipients may act to facilitate incorporation of the therapeutically active agent onto or into the substrate, modify the release of the therapeutically active agent from the substrate, stabilize the therapeutically active agent, or enhance absorption of the therapeutically active agent. Pharmaceutical excipients are disclosed in “Remington's Pharmaceutical Sciences,” 17ₜₕ Ed (1985), pp.1603-1644, which is incorporated herein by reference. The formulation of the therapeutically active agent and the excipients is selected according to criteria well known to those skilled in the art to achieve the desired release rate, stability, absorption and facilitation of dosage form manufacture. Excipients in solid formulations include, but are not limited to, diluents, binders, stability enhancers, lubricants, disintegrants, colors, flavors, and sweeteners. Solvents may be considered as excipients but will be eliminated in the final form.

Suitable binders for use in the present formulation include but are not limited to synthetic gums such as hydroxypropyl methylcellulose (“HPMC”), hydroxypropyl cellulose (“HPC”,e.g. Klucel™), carboxymethylcellulose, ethylcellulose and methylcellulose, starch, gelatin sugars and natural gums, preferably hdroxypropyl cellulose (e.g. Klucel™).

Suitable solvents for use in the present formulation are non-aqueous solvents, and include but are not limited to dehydrated alcohols, preferably ethanol (200 proof).

Another suitable excipient for use in the present formulation is talc added to reduce the stickiness of coating formulations. The talc particles are very easily embedded in the polymer layers, thus significantly reducing sticking during the film forming process. Talc also reduces the porosity of film coating and lowers their water permeability.

“Enteric polymers” means polymers which remain insoluble in the stomach, but dissolve at higher pH of the intestine, are used to deliver drugs to the small intestine. Enteric coating also prevents drugs from degradation by gastric fluid and enzymes. Enteric polymers include, but are not limited to cellulose acetate phthalate, hydroxypropylcellulose acetate phthalate, polyvinyl acetate phthalate, methacrylate-methacrylic acid copolymers, styrol, maleic acid copolymers, shellac, Eudragi™ preferably but not limited to the L or S series, and others.

“Hydrophobic” refers to the property of a substance that is substantially repellant to water.

“Optional” or “optionally” means that the subsequently described event or circumstance may but need not occur, and that the description includes instances where the event or circumstance occurs and instances in which it does not.

“Labile” means that a linker group, under the appropriate physiological conditions, will be rapidly and efficiently broken down, thus decomposing the compound.

“Core” means a starter material for pellet preparation deemed to encompass spheres, seeds, pellets, spheroids, granules, beads, particles, and the like. Examples of cores include, but are not limited to sugar spheres (non-pareils, neutral pellets, sugar spheres, Nu-Pareil, Nu-Core, sugar seeds.) or microcrystalline cellulose spheres Celphere®, most preferably sugar spheres. Sugar spheres are approximately spherical granules of a labeled nominal-size range with a uniform diameter and containing not less than 62.5% and not more than 91.5% of sucrose, calculated on the dried basis. The remainder is chiefly starch.

Processes for the preparation of compounds of Formula I and of N-[6-chloro-3-( 4,5-dihydro-1H-imidazol-2-ylmethoxy)-2-methyl-phenyl]-methanesulfonamide are disclosed in U.S. Pat. No.5,952,362.

Description

According to the well known methods in the art, a number of contemporary pharmaceutical solid-dosage form processing trains including but not limited to extrusion/spheronization, spray drying and fluidization, preferably fluidization can be carried out. Spherical cores (composition as per US Pharmacopeia, preferably non-pareils) are coated preferably in a fluidized bed, with a first layer that comprises an enteric polymer such as acrylic polymers, alkylcelluloses and mixtures thereof, and optional hydrophobic excipients in a non-aqueous solvent such as alcohol. A preferred excipient is talc, preferred polymers are shellac or Eudragit™(preferably Eudragit L or S). After the drying of the first layer, the second layer that comprises one or more labile pharmaceutically active compounds in one or more acceptable hydrophobic excipients in a non-aqueous solvent such as alcohol is sprayed on the first coating by conventional fluidized bed coating techniques. Preferred excipients comprise hydroxypropyl cellulose, e.g., Klucel EXF, or Eudragit™, preferably but not limited to series RS 100 with talc. Optionally a third layer that comprises an enteric polymer in a non-aqueous solvent, providing further stabilization, or allowing delayed or sustained release, is sprayed onto the second coating layer comprising the labile drug. A preferred polymer for the third layer is Eudragit™, preferably but not limited to series RS 100.

The pharmaceutical spheres of the present invention can be readily formulated per se or in combination with a conventional appropriate carrier into a delivery form such as, but not limited to, capsules or pellets.

### EXAMPLE

In 1311.7 g of alcohol (200 proof), 1186.0 g of refined pharmaceutical glaze, per National Formulary (NF) and 131.0 g of talc, per US Pharmacopeia (USP) were added and mixed until a uniform dispersion was obtained. 3947.4 g of sugar spheres, NF were added to a fluidized bed apparatus and the suspension was sprayed on the spheres. The spheres were dried before applying the second layer.

In 673.1 g of alcohol (200 proof), 24.8 g of hydroxypropyl cellulose, NF, 83.7 g of talc, USP and 50.0 g of micronized active compound of Formula I , wherein A is —OCH₂—, R¹ and R⁶ are methyl, R³ is chloro, and R², R⁴ and R⁵ are hydrogen, named N-[6-chloro-3-(4,5-dihydro-1H-imidazol-2-ylmethoxy)-2-methyl-phenyl]-methanesulfonamide, were dispersed. This dispersion was sprayed onto the spheres obtained from the first step and dried.

When needed, a third spraying step in which a dispersion with glaze and talc (identical to the first dispersion) was sprayed on the spheres coated with drug for additional stabilization, or allowing delayed or sustained release.

The coated spheres were filled into hard gelatin capsules and stored at 25° C. and 60% relative humidity in high density polyethylene bottles. The degradation in the above capsules (expressed as percent of hydrolysis product deriving from the decomposition of N-[6-chloro-3-(4,5-dihydro-1H-imidazol-2-ylmethoxy)-2-methyl-phenyl]-methanesulfonamide) was compared to the degradation in conventional tablets, prepared by the traditional wet granulation process and stored similarly. The results are shown in Table 1. The non-pareil capsule formulation showed lower levels of the hydrolysis product over extended periods of time compared to the tablets prepared by the conventional process using conventional excipients.

TABLE 1
Stability of non-pareil formulation compared to tablets
prepared by traditional wet granulation and stored at 25° C.
and 60% relative humidity

| | % of hydrolysis product at | | |
|---|---|---|---|
| Formulation | Initial | 1 month | 3 months |
| Tablet | 0.34 | 1.02 | 1.72 |
| Capsule filled with | 0.50 | 0.59 | 0.33 |
| non-pareils | | | |

What is claimed is:

1. A stabilized oral pharmaceutical formulation comprising:
a) a nucleus formed by a core;
b) a first layer that comprises a polymer coating sealing the core and optionally one or more hydrophobic excipients; and
c) a second layer coating the first layer, wherein said second layer comprises one or more labile pharmaceutically active compounds in one or more acceptable hydrophobic excipients.

2. The pharmaceutical formulation of claim 1, wherein the polymer coating comprises an enteric polymer.

3. The pharmaceutical formulation of claim 2, wherein the polymer coating comprises shellac or Eudragit™ (L or S series).

4. The pharmaceutical formulation of claim 3, wherein the one or more labile pharmaceutically active compounds in the second layer are susceptible to hydrolytic degradation.

5. The pharmaceutical formulation of claim 3, wherein the labile pharmaceutically active compound in the second layer is a compound comprising an imidazoline moiety.

6. The pharmaceutical formulation of claim 5, wherein the labile pharmaceutically active compound in the second layer is a compound of Formula Ar—A—B, wherein Ar is a substituted aryl group, A is —NH—, —CH₂—,or —OCH₂—, and B is 2-imidazoline.

7. The pharmaceutical formulation of claim 6, wherein the labile pharmaceutically active compound in the second layer is a compound of Formula I: 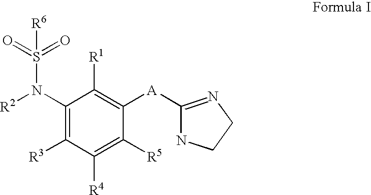
wherein:
A is —NH—, —CH₂—, or —OCH₂—;
R¹, R³, R⁴, and R⁵ are each independently in each occurrence hydrogen, (C₁-C₆) alkyl, or halogen;
R⁶ is (C₁-C₆) alkyl;
R² is hydrogen or (C₁-C₆) alkyl; or
R² and R³ taken together with the atoms to which they are attached may form a 5- or 6-membered ring;
or pharmaceutically acceptable salts thereof.

8. The pharmaceutical formulation of claim 7, wherein the labile pharmaceutically active compound is a compound of Formula I, wherein A is —OCH₂—, R¹ and R⁶ are methyl, R³ is chloro, and R², R⁴ and R⁵ are hydrogen, named N-[6-chloro-3-(4,5-dihydro-1H-imidazol-2-ylmethoxy)-2-methyl-phenyl]-methanesulfonamide.

9. The pharmaceutically composition of claim 1, wherein the labile pharmaceutically active compound in the second layer is N-[6-chloro-3-(4,5-dihydro -1H-imidazol-2-ylmethoxy)-2-methyl-phenyl]-methanesulfonamide.

10. The pharmaceutical formulation of claim 1, further comprising a third layer coating the second layer, wherein the third layer is an enteric polymer.

11. The pharmaceutical formulation of claim 7, further comprising a third layer coating the second layer, wherein the third layer is an enteric polymer.

12. The pharmaceutical formulation of claim 8, further comprising a third layer coating the second layer, wherein the third layer is an enteric polymer.

13. A process for the formulation of a stable oral pharmaceutical formulation of claim 1, which process comprises:
a) coating a core with a first layer sealing the core, wherein said first layer comprises an enteric polymer layer optionally comprising one or more hydrophobic excipients in an non-aqueous solvent.
b) drying the first layer;
c) coating the first layer with a second layer, wherein said second layer comprises one or more pharmaceutically active labile compounds, suspended in one or more acceptable hydrophobic excipients;
d) drying the second layer,
e) optionally coating the second layer with a third layer, wherein said third layer comprises an enteric polymer in a non-aqueous solvent, and
f) drying the third layer.

14. The process of claim 13, wherein the labile pharmaceutically active compound is a compound of Formula I: 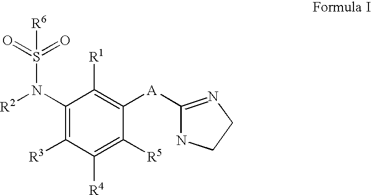
wherein:
A is —NH—, —CH₂—, or —OCH₂—;
R¹, R³, R⁴, and R⁵ are each independently in each occurrence hydrogen, (C₁-C₆) alkyl, or halogen;
R⁶ is (C₁-C₆) alkyl;
R² is hydrogen or (C₁-C₆) alkyl, or
R² and R³ taken together with the atoms to which they are attached may form a 5- or 6-membered ring;
or pharmaceutically acceptable salts thereof.

15. The process of claim 14, wherein the labile pharmaceutically active compound is a compound of Formula I, wherein A is —OCH₂—, R¹ and R⁶ are methyl, R³ is chloro, and R², R⁴ and R⁵ are hydrogen, named N-[6-chloro-3-( 4,5-dihydro-1H-imidazol-2-ylmethoxy)-2-methyl-phenyl]-methanesulfonamide.

16. A method of treatment of urinary incontinence comprising administering a stable oral pharmaceutical formulation according to claim 7.

17. A method of treatment of urinary incontinence comprising administering a stable oral pharmaceutical formulation according to claim 8.

18. A method of treatment of urinary incontinence comprising administering a stable oral pharmaceutical formulation according to claim 9.

19. A method of treatment of urinary incontinence comprising administering a stable oral pharmaceutical formulation according to claim 11.

20. A method of treatment of urinary incontinence comprising administering a stable oral pharmaceutical formulation according to claim 12.

21. The method of treatment of claim 16 comprising administering the stable oral formulation in capsules or pellets.

22. The method of treatment of claim 17 comprising administering the stable oral formulation in capsules or pellets.

23. The method of treatment of claim 18 comprising administering the stable oral formulation in capsules or pellets.

24. The method of treatment of claim 19 comprising administering the stable oral formulation in capsules or pellets.

25. The method of treatment of claim 20 comprising administering the stable oral formulation in capsules or pellets.
